(19) United States
(12) Patent Application Publication
(10) Pub. No.: US 2026/0367701 A1
(43) Pub. Date: Nov. 19, 2026

(54) METHOD, COMPUTER PROGRAM AND SYSTEM FOR DETERMINING INLIERS OF GAZE POSITIONS OF A PERSON

(71) Applicant: MACHINEMD AG, Bern (CH)

(72) Inventors: Sareh SAEEDI, Neuchâtel (CH); Romain GAULIER, Neuchâtel (CH); Kishan SHARMA, Neuchâtel (CH); Engin TÜRETKEN, Neuchâtel (CH); Andrea DUNBAR, Neuchâtel (CH); Dominik BRÜGGER, Bern (CH)

(73) Assignee: MACHINEMD AG, Bern (CH)

(21) Appl. No.: 19/515,478

(22) PCT Filed: Aug. 29, 2024
(86) PCT No.: PCT/EP2024/074174

§ 371 Date: Mar. 1, 2026

(30) Foreign Application Priority Data
Aug. 29, 2023 (EP) 23194115.4
Aug. 30, 2023 (EP) 23194392.9

(51) Int. Cl. G06F 3/01; G06V 40/18

(52) U.S. Cl. CPC G06F 3/013; G06V 40/193

(57) ABSTRACT

The invention relates to a method for determining inliers (1) of gaze positions (2) of a person, wherein the method comprises the steps of: 1) displaying an optical stimulus (3) in a visual field (4) of the person, 2) detecting a time series of gaze positions (2) of the person in the visual field (4) in response to the optical stimulus (3), 3) determining inliers (1) of the detected gaze positions (2), wherein the inliers (1) are detected gaze positions (2) that are located within a first range of positions (5), wherein a number of detected gaze positions (2) in the first range (5) is greater than a number of detected gaze positions (2) that are located within any other range of the same given size as the first range (5), or wherein the inliers (1) are detected gaze positions (2) that are located within a second range of positions (50), wherein a number of detected gaze positions (2) in the second range (50) amounts to at least 0.5 times the number of gaze positions (2) in the first range (5), wherein the first and second range of positions (5,50) are found using continuous or discrete optimization techniques or wherein the first and second range of positions (5,50) are found by a sampling technique, wherein the optical stimulus (3) is subsequently displayed and located for a predetermined time at a plurality of different stimulus positions (8,8a,8b,8c) in the visual field (4) of the person, and wherein for at least some of the stimulus positions (8,8a,8b,8c), respective inliers (1) are determined from respective time series of gaze positions (2). The invention further relates to a computer program and a system (10) for determining inliers (1) of gaze positions (2) of a person.

The present invention relates to a method for determining inliers of gaze positions of a person, a computer program for determining inliers of gaze positions of a person and a system for determining inliers of gaze positions of a person.

Particularly in human-computer interaction and physiological research, the accurate and reliable determination of gaze positions plays a pivotal role. Gaze tracking systems have evolved significantly, enabling a broad spectrum of applications, ranging from immersive augmented or virtual reality experiences to enhancing our understanding of sensory and motor functions of the eye as well as cognitive processes. However, these advancements often bring about challenges related to the identification of reliable gaze position data from measurements that comprise relatively strong positional changes of the gaze, as well as noise or measurement errors.

Conventional gaze tracking methods often struggle with the identification of so-called inliers, that is, a subset of raw gaze positions that are considered as accurately representing the subject's true visual focus, within a stream of captured data. One of the prominent challenges in this domain is posed by the inherently dynamic nature of human gaze. Positional changes of gaze, driven not only by saccadic movements or subtle shifts in attention but also by movements between the eye and a head-mounted display system worn by the person, can often lead to false results. These changes introduce inaccuracies that hinder the overall reliability of gaze tracking systems. The problem is exacerbated in scenarios involving subjects with spontaneous eye movements, such as individuals with certain medical conditions or children who exhibit greater variability in gaze behavior. US2020125166A1 proposes technologies for performing user-specific calibration of eye tracking systems for Near-Eye-Display (NED) devices. The NED device may sequentially present different virtual stimuli to a user while concurrently capturing instances of eye tracking data. The eye tracking data reveals calibration ellipse centers that uniquely correspond to individual virtual stimuli. However, in this approach, the stimuli positions themselves are used as calibration centers for determining eye tracking data and inliers of detected gaze positions are determined by means of detected gaze positions that are located within a predefined threshold distance around the respective stimuli position. The inliers determined in that fashion are thus not necessarily representative for the real gaze of a person. Inliers determined according to this approach are in particular not robust against blink events or eye movements such as saccades.

Based on this, it is subject of the present invention to provide a method, a computer program and a system for determining inliers of gaze positions of a person, that are each improved in that they allow for a determination of inliers that is more robust against spatial variations of the detected gaze positions compared to the prior art.

Such method, computer program and system can be used to determine inliers of raw gaze positions detected and output by a head-mounted display system in order to provide more robustness against spatial variations and noise in the raw gaze positions compared to the prior art.

The gaze positions may be particularly raw gaze positions of a person, i.e. unprocessed data indicative of gaze positions generated by a gaze detection algorithm.

This task is solved by a method for determining inliers of gaze positions of a person with the features of claim 1, a computer program for determining inliers of gaze positions of a person according to claim 17 as well as a system for determining inliers of gaze positions of a person according to claim 18.

Advantageous embodiments of the invention are given in the corresponding dependent claims and described in the following.

A first aspect of the invention relates to a method for determining inliers of gaze positions of a person. The method comprises the following steps:
i) displaying an optical stimulus in a visual field of the person,
ii) detecting a time series of gaze positions of the person in the visual field in response to the optical stimulus,
iii) determining inliers of the detected gaze positions, wherein the inliers are detected gaze positions of the time series of gaze positions that are located within a first range of positions, wherein a number of detected gaze positions in the first range of positions is greater than a number of detected gaze positions that are located within any other range of the same given size as the first range, or wherein the inliers are detected gaze positions that are located within a second range of positions of the time series of gaze positions, wherein a number of detected gaze positions in the second range of positions amounts to at least 0.5 times the number of gaze positions in the first range of positions, wherein the first and second range of positions are found using continuous or discrete optimization techniques or wherein the first and second range of positions are found by a sampling technique.

The visual field of the person can be defined in one-dimensional, two-dimensional or three-dimensional space, and in any coordinate system such as spherical, cylindrical, polar or cartesian coordinate systems. For instance, an optical stimulus or a detected gaze position can be specified by its vertical and horizontal positions or -angles, for example on a metric scale or in degrees in a two-dimensional cartesian space, e.g., a point on a screen, or three numbers representing a radial distance from an origin, the polar angle and the azimuthal angle in a three-dimensional spherical coordinate system, e.g., a point in a 3D virtual word.

For a given size of the first range of positions, the first range of positions comprises the highest number of detected gaze positions within the time series of detected gaze positions, such that the inliers detected this way are particularly representative for the visual focus of the person in response to the optical stimulus. At the same time, the inliers determined with respect to the first range of positions are particularly robust against spatial variations of the gaze, as the number of inliers is maximized.

The given size of the first and the second range of positions can be defined for example as a two-dimensional area in the visual field, for example by two-dimensional same given size shapes in the visual field of the person, such as circles, ellipses, squares, rectangles and the like, in order to include a two-dimensional range of positions of a portion in the visual field of the person. However, the first and the second range may also be considered as three-dimensional or one-dimensional ranges. For instance, as a one-dimensional example, one can consider a vertical range of positions corresponding to a vertical component of the visual field of the person and/or a horizontal range of positions. One-dimensional ranges can also be referred to as intervals. The one-dimensional ranges can be projections of a two-dimensional range onto the two orthogonal axes spanning the two-dimensional space.

The term “given size” can be interpreted as a given input size for the method according to the invention. That means that the given input size is used as an input value, and that the first range of positions is determined as the one range that comprises a number of detected gaze positions that is greater than a number of detected gaze positions that are located in any other range of the same given input size. The same can be applied for the second range with the number of detected gaze positions amounting to at least 0.5 times the number of gaze positions in the first range. Hence, the continuous or discrete optimization techniques or sampling techniques do not aim at determining the size of the first range, but uses a given size as an input value in order to find the locations or absolute positions in space—i.e. the first range—that comprises the highest number of detected gaze position. This allows for a computational efficient and accurate determination of inliers that can be identified as the detected gaze positions within said first range.

Particularly, the term “same given size” may refer to a range having the same given size and shape, particularly including the same orientation. This notion of the term “same given size” may allow a suitable interpretation in case the range is a two-dimensional or a three-dimensional dimensional range.

In other words, in case the range is a one-dimensional range, e.g. in case the gaze positions are determined along one coordinate only, the term “same given size” particularly refers to the given size of an interval.

According to the invention, the inliers are detected gaze positions that are located within a first range of positions, wherein a number of detected gaze positions in the first range of positions is greater than a number of detected gaze positions that are located within any other range of the same given size as the first range of positions. Such a first and second range of positions can be found by using continuous or discrete optimization techniques or sampling techniques such as a random sample consensus (RANSAC), exhaustive discrete optimization, grid search or the Mean-Shift clustering algorithms. For example, if exhaustive discrete optimization is preferred, for each detected gaze position, said geometrical shape, such as a circle in a two-dimensional space, can be centered around the respective detected gaze position, and the number of detected gaze positions within the geometrical shape centered around the respective gaze position is counted. The particular gaze position, which is associated with the highest number of detected gaze positions, represents the first range of positions.

In particular, if the first and second range of positions are found using the sampling technique, the sampling technique comprises sampling the detected gaze positions in a plurality of ranges of positions, particularly in all ranges of positions, to identify the first range of positions or the second range of positions. In other words, for a plurality of ranges, particularly for all ranges, a respective number of detected gaze positions that fall into the respective range is counted and the first range is identified as the one range that comprises the highest number of detected gaze positions of the plurality of ranges, particularly of all ranges.

The different ranges for sampling can be based on the detected gaze positions. In a one-dimensional example, the ranges can be sampled starting from the lowest detected gaze position to another detected gaze position, wherein the range has said given size, for instance—in a one-dimensional example—the width of a one-dimensional interval. The number of detected gaze positions within each range is counted. This process is repeated for each range, moving from the lowest to the highest detected position, until all detected gaze positions have been sampled. The one range with the highest number of detected gaze positions is identified as the first range. Accordingly, the second range of positions can be identified as ranges that comprise a number of detected gaze positions amounting to at least 0.5 times the number of gaze positions in the first range.

As ranges, the first and the second range of positions include absolute positional information of the gaze positions within the first and the second range of positions. In other words, no reference position for the range in the visual field needs to be specified to fully define the portions or intervals of the visual field comprising the first and/or the second range of positions, as they comprise absolute positional information.

Particularly, the inliers may also be selected from detected gaze positions that are located within the second range of positions, wherein said number of detected gaze positions in the second range of positions amounts to at least 0.5 times the number of gaze positions in the first range of positions. While this approach leads to slightly less robust results compared to the inliers located within the first range of positions, the inliers associated to the second range of positions still yield reliable information about the visual focus of the person in response to the optical stimulus.

For example, the optical stimulus comprises or consists of symbol characters, letters, numbers or at least sections of geometrical shapes, such as crosses, rectangles, circles, squares, blobs, points or the like. The optical stimulus can also comprise or consist of more complex virtual or real (digitized) objects such as a bird, a planet, or a photograph. The optical stimulus can be displayed in front of a background, that may represent the actual physical environment in the visual field of the person, particularly as an augmented reality feature. However, optical stimulus may also be displayed in front of a virtual background forming the visual field. Particularly, the optical stimulus is displayed in front of a uniform background of constant luminosity and/or color, for example in front of a grey background. Alternatively, the optical stimulus can be displayed in a virtual environment, for example wherein the optical stimulus is displayed in front of a virtual landscape.

Particularly, prior to the determination of the inliers, the time series of gaze positions of the person in the visual field in response to the optical stimulus is filtered, for example to remove blink events of the person.

According to an embodiment of the invention, the number of detected gaze positions in the second range of positions amounts to at least 0.7 times, particularly 0.9 times the number of gaze positions in the first range of positions. The inliers determined according to this embodiment are more robust against spatial variations of the detected gaze positions compared to the case where the number of detected gaze positions in the second range of positions amounts to at least 0.5 times the number of gaze positions in the first range of positions.

In an embodiment of the invention, detected gaze positions that are not comprised by the first range of positions or the second range of positions define outliers and wherein at least some of the inliers are temporally separated by outliers. The outliers can be associated to spatial variations of the detected gaze positions, for example related to eye blinks, saccadic movements, which are movements between a head mounted display for displaying the optical stimulus to the person and the eye of the person, or failures or imperfections of a gaze detection algorithm or the head mounted display. The outliers can thus be effectively separated from the inliers, such that the visual focus of the person can be determined even for relatively strong spatial variations of the detected gaze positions, which is particularly advantageous if inliers are to be determined for persons with medical conditions or children, who typically exhibit a greater variability in gaze behavior.

In an embodiment of the invention, the given size of the first range of positions is based on a positional spread of the detected gaze positions. Among other things, the positional spread of the detected gaze positions can be related to eye blinks or saccadic movements of the eye of the person. For example, the positional spread can be quantified by means of a positional variance of gaze positions comprised by the time series of gaze positions.

According to yet another embodiment, the given size of the first range of positions is larger for larger positional spread compared to smaller positional spread. In case the variance is taken as a quantitative measure for the positional spread, the range of positions can be larger for larger variance compared to smaller variance of the detected gaze positions.

In an embodiment of the invention, during display, the optical stimulus is located for a predetermined time at a predefined stimulus position in the visual field of the person during detection of the time series of gaze positions. In other words, the optical stimulus is kept at the same predefined stimulus position during detection of the time series of gaze positions. As such, the person does not need to react to further positional changes of the optical stimulus during the detection of the time series associated with the predefined stimulus position, such that the inliers determined from the time series is particularly representative for the visual focus of the person to a specific stimulus position.

According to an embodiment of the invention, the optical stimulus is subsequently displayed and located for a predetermined time at a plurality of different stimulus positions in the visual field of the person, and wherein for at least some of the stimulus positions, respective inliers are determined from respective time series of gaze positions according to the invention. For example, the optical stimulus is displayed at different positions of a predetermined pattern of stimuli positions in the visual field of the person, for example a regular grid with equidistantly or equiangularly spaced stimulus positions that form and fill a square, a rectangle, a circle, an ellipse, or a diamond-shape in the visual field of the person. For example, the number of different stimulus positions is between 1 and 100, particularly between 1 and 30.

In another embodiment of the invention, for at least one stimulus position, an aggregate gaze coordinate, particularly an averaged gaze coordinate, is determined from the inliers associated to the stimulus position. The aggregate gaze coordinate represents a single coordinate in the visual field of the person and summarizes the plurality of the inliers obtained in response to the display of the optical stimulus at a specific stimulus position. To this end, for example, the aggregate gaze coordinate can be calculated as a median or average positional value of the inliers associated to the stimulus position. The aggregate gaze coordinate can also comprise other features such as the variance of the inliers, or the number of inliers, in addition to the median or average positional value of the inliers. If the detected gaze positions are attached with confidence values, the averaging or median filtering can also take these confidences into account to compute a weighted average or a weighted median.

In yet another embodiment of the invention, for at least one stimulus position, a spatial displacement, particularly a distance between the stimulus position and the aggregate gaze coordinate associated with the stimulus position is determined. The accuracy of the determined aggregate gaze coordinate can vary for multiple reasons, such as too strong spatial variations of the detected gaze coordinates as a consequence of distraction of the person or impairments in the field of view of the person, but also technical issues of a head mounted display used for the display of the optical stimuli.

In an embodiment of the invention, a subsequent stimulus position is based on the spatial displacement between a preceding stimulus position and the aggregate gaze coordinate associated with the preceding stimulus position. In other words, subsequent stimulus positions can be adapted according to aggregate gaze coordinates determined for preceding stimulus positions. For example, if for the preceding stimulus position and the associated aggregate gaze coordinate, the displacement exceeds a maximum displacement, at least one of the succeeding stimulus positions can be arranged in a vicinity of the preceding stimulus position or again at the preceding stimulus position. This measure represents a confirmation measurement that can confirm whether the aggregate gaze coordinate associated to the preceding stimulus position was based on an event such as distraction of the user or movements between the head mounted display and the eye of the person, or on systematic problems such as impairments in the visual field of the person or functional issues of the head mounted display. Succeeding stimulus positions can also be based on the spatial displacement determined between preceding stimulus positions and their respective associated aggregate gaze coordinate in that if a trend between stimulus positions and their associated spatial displacements is found. For example, if for stimulus positions with larger distances to an origin of the visual field the spatial displacement to their respective aggregate gaze coordinates is larger than for stimulus positions with smaller distances to the origin of the visual field, at least some of the succeeding stimulus positions can be arranged closer to the origin of the visual field compared to the stimulus positions with larger distances to the origin of the visual field. For example, the origin of the visual field is arranged at a center of area of the visual field. This measure particularly allows to determine reliable inliers for multiple positions in the visual field even in case of impairments of the visual field that may be for instance in a peripheral area of the visual field.

Alternatively, the succeeding stimulus position can be determined based on the number and/or the spatial spread of the inliers associated to preceding stimulus position. For example, if the number of inliers is smaller than a minimum value for the preceding stimulus position, at least one of the succeeding stimulus positions can be arranged in a vicinity of the preceding stimulus position or again at the preceding stimulus position.

In another embodiment of the invention, a transformation model is generated that links each stimulus position with the associated aggregate gaze coordinate. The term “link” particularly refers to an estimation of each stimulus position from its associated aggregate gaze coordinate. The transformation model can provide a calibration between each displayed stimulus position and the associated aggregate gaze coordinate, which allows for a spatial correction of contents to be displayed in the visual field of the person using for example a head mounted display, which is particularly relevant for augmented- and/or virtual reality applications. For example, the transformation model can be based on polynomials that establish an analytical connection between each displayed stimulus position and the associated aggregate gaze coordinate. The transformation model can also be based on homography or neural networks. Since the determination of the aggregate gaze coordinate based on the inliers yields a single aggregate gaze coordinate associated to each stimulus position, the transformation model is substantially simplified compared to methods of the prior art that generate the transformation model based on time series of detected gaze positions for each stimulus position. For example, if polynomials are used for the transformation model, a second order polynomial is typically sufficient to link each stimulus position to the associated aggregate gaze coordinate, which substantially reduces the time needed to generate the transformation model. In the estimation of the transformation model, if the aggregate gaze coordinates comprise also other features such as the variance of the inliers or the number of inliers, these features can be used to estimate the transformation model more reliably. For instance, in optimization cost function for the model parameters, an aggregate gaze coordinate with a smaller variance or a larger number of inliers can be weighted more heavily than an aggregate gaze coordinate with a larger variance or a smaller number of inliers.

According to another embodiment of the invention, stimulus positions that are associated with a spatial displacement that exceeds said maximum spatial displacement, are not considered for the generation of the transformation model. For example, the maximum distance can be defined as a distance offset or threshold plus the median or average distance between the stimulus positions and their respective associated aggregate gaze coordinate, wherein a stimulus position and its respective associated aggregate gaze coordinate are discarded and thus not considered for the generation of the transformation model if their distance exceeds the maximum distance. Likewise, the maximum distance can also be defined as a certain multiplicative factor (e.g., 1.5) of the median or average distance between the stimulus positions and their respective associated aggregate gaze coordinates.

According to another embodiment of the invention, upon the generation of the transformation model, the optical stimulus can be displayed at a plurality of predetermined validation positions. The optical stimulus is subsequently displayed at the validation positions and respective time series of gaze positions in response to the display of the optical stimulus at the different validation positions are determined. From the respective time series, respective inliers and particularly respective aggregate gaze coordinates may be determined. From pairs of the respective validation positions and their associated aggregate gaze coordinates, a mean validation accuracy indicative of the efficiency of the transformation model may be determined. The validation accuracy can comprise an average displacement error between the validation positions and the model-transformed positions of their associated aggregate gaze coordinates. In particular, validation points with essentially the same or similar distance to the origin of the visual field can be grouped and their respective displacement errors to their associated aggregate gaze position can be averaged. This procedure can be performed for groups of validation positions with different distances to the origin of the visual field, in order to generate information about the transformation accuracy for validation points with different distances to the origin, and hence different field-of-views of the visual field.

According to another embodiment of the invention, a map is generated that relates at least one stimulus position to the detected gaze positions associated to the stimulus position. In particular, the respective aggregate gaze coordinate associated to the at least one stimulus position is represented by the map. Moreover, said transformation accuracy can be indicated on said map, such that the efficiency of the transformation model can be read off on a spatial representation of the visual field.

In an embodiment of the invention, the optical stimulus is applied to only one eye of the person and wherein the inliers are determined for the one eye or for both eyes separately. Particularly, the respective inliers detected for both eyes are compared, which delivers useful information about the alignment or misalignment of the visual axes of the eyes. Typically, for healthy persons, if the optical stimulus is applied to a single eye of the person, a saccadic movement in reaction to the display of the optical stimulus to the single eye is not just triggered in the eye exposed to the optical stimulus, but also to the other eye that is not exposed to the optical stimulus. In particular, the optical stimulus is first applied to a first eye of the person with the second eye not being exposed to the optical stimulus and the inliers are determined for one of both eyes or for both eyes separately, whereafter the optical stimulus is applied to the second eye of the person with the first eye not being exposed to the optical stimulus and the inliers are determined for one of both eyes or for both eyes separately.

A second aspect of the invention relates to a computer program for determining inliers of gaze positions of a person. The computer program comprises instructions which, when the program is executed by a computer, cause the computer to:
cause an optical device to
   display an optical stimulus in a visual field of the person,
   detect a time series of gaze positions of the person in the visual field in response to the optical stimulus
determine inliers of the detected gaze positions, wherein the inliers are detected gaze positions that are located within a first range of positions, wherein a number of detected gaze positions in the first range of positions is greater than a number of detected gaze positions that are located within any other range of the same given size as the first range of positions, or wherein the inliers are detected gaze positions that are located within a second range of positions, wherein a number of detected gaze positions in the second range of positions amounts to at least 0.5 times the number of gaze positions in the first range of positions, wherein the first and second range of positions are found using continuous or discrete optimization techniques or wherein the first and second range of positions are found by a sampling technique.

In other words, the computer is configured to execute the computer-implemented steps of the method according to the first aspect of the invention. The embodiments of the first aspect of the invention can therefore also be applied to the second aspect of the invention.

A third aspect of the invention relates to a system for determining inliers of gaze positions of a person. The system comprises:
an optical device configured to:
   display an optical stimulus in a visual field of the person,
   detect a time series of gaze positions of the person in the visual field in response to the optical stimulus, and
the computer according to the second aspect of the invention.

Since the system according to the third aspect of the invention comprises the computer according to the second aspect of the invention, which is configured to execute the computer-implemented steps of the method according to the first aspect of the invention, the embodiments of the first aspect of the invention can also be applied to the third aspect of the invention.

Particularly, the optical device of the system according to the third aspect of the invention is integrated in goggles, particularly in a head-mounted display to be worn by a person. The computer may or may not be integrated in the goggles.

The optical device can receive data indicative of commands of the computer that cause the optical device to display the optical stimulus in the visual field of the person and to communicate with the computer by sending data indicative of the detected gaze positions of the person to the computer, such that the computer can determine the inliers of the detected gaze positions.

To this end, if the computer is integrated in the goggles, the optical device and the computer can communicate for example via cable-based communication means. If the computer is not integrated in the goggles, the optical device and the computer can communicate via wired or wireless communication means.

In particular, data indicative of the detected gaze positions of the person can be sent to a server or a cloud for determination of the inliers by means of the cloud or the server.

An item of the invention relates to a method for determining inliers of gaze positions of a person. The method comprises the following steps:
i) displaying an optical stimulus in a visual field of the person,
ii) detecting a time series of gaze positions of the person in the visual field in response to the optical stimulus,
iii) determining inliers of the detected gaze positions, wherein the inliers are detected gaze positions of the time series of gaze positions that are located within a first range of positions, wherein a number of detected gaze positions in the first range of positions is greater than a number of detected gaze positions that are located within any other range of the same size as the first range, or wherein the inliers are detected gaze positions that are located within a second range of positions of the time series of gaze positions, wherein a number of detected gaze positions in the second range of positions amounts to at least 0.5 times the number of gaze positions in the first range of positions, particularly wherein the first and second range of positions are found using continuous or discrete optimization techniques or wherein the first and second range of positions are found by a sampling technique.

Exemplary embodiments are described below in conjunction with the Figures. The Figures are appended to the claims and are accompanied by text explaining individual features of the shown embodiments and aspects of the present invention. Each individual feature shown in the Figures and/or mentioned in the text of the Figures may be incorporated (also in an isolated fashion) into a claim relating to the first aspect, the second aspect and/or the third aspect according to the present invention.

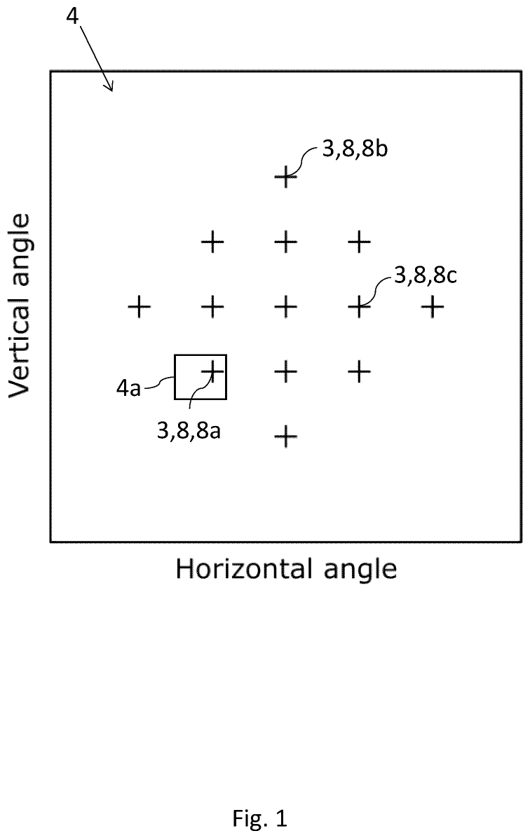

FIG. 1 shows a plurality of optical stimuli displayed at different positions in a visual field of a person according to an embodiment of the invention, for determining inliers of gaze positions of a person in response to the optical stimulus;

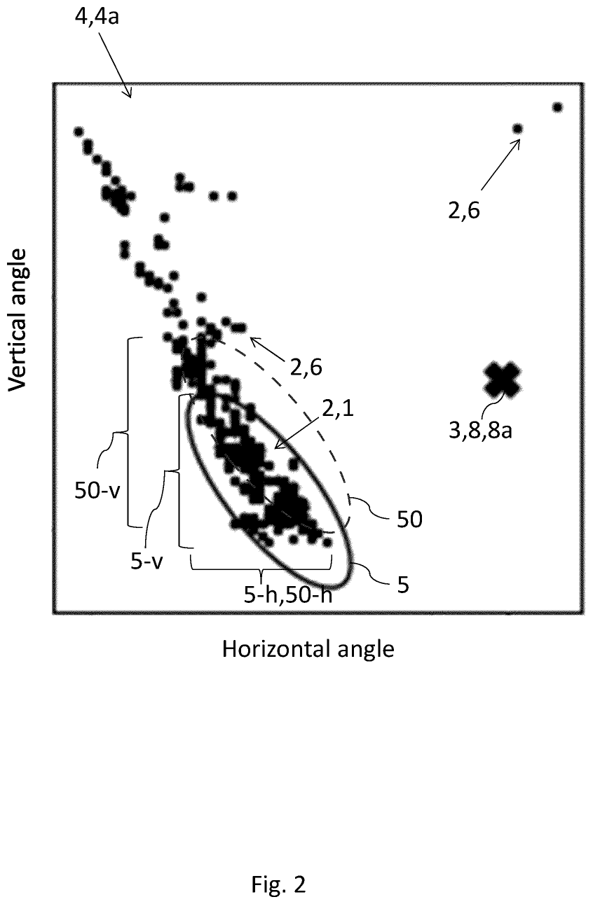

FIG. 2 shows an embodiment of the method according to the invention, highlighting a first time series of gaze positions detected in the visual field of the person in response to the displaying of the optical stimulus at a first stimulus position shown in FIG. 1;

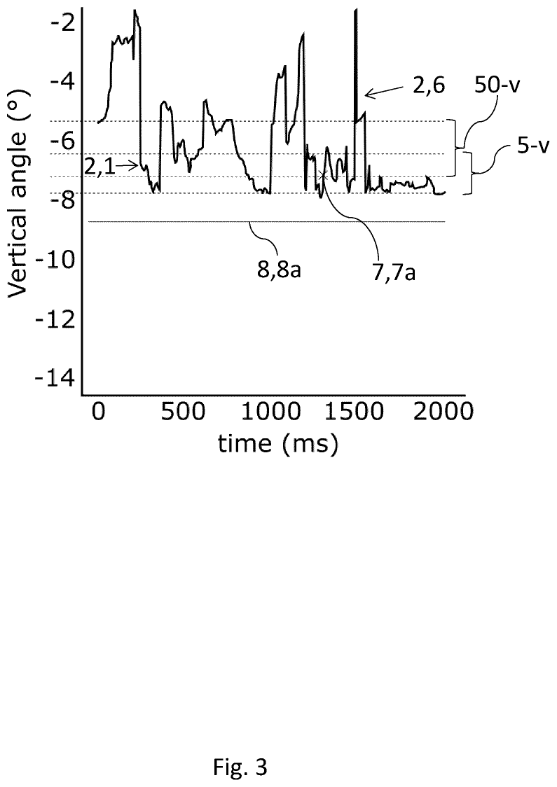

FIG. 3 shows an embodiment of the method according to the invention, wherein an aggregate gaze coordinate is determined from inliers of the first time series of detected gaze positions of FIG. 2;

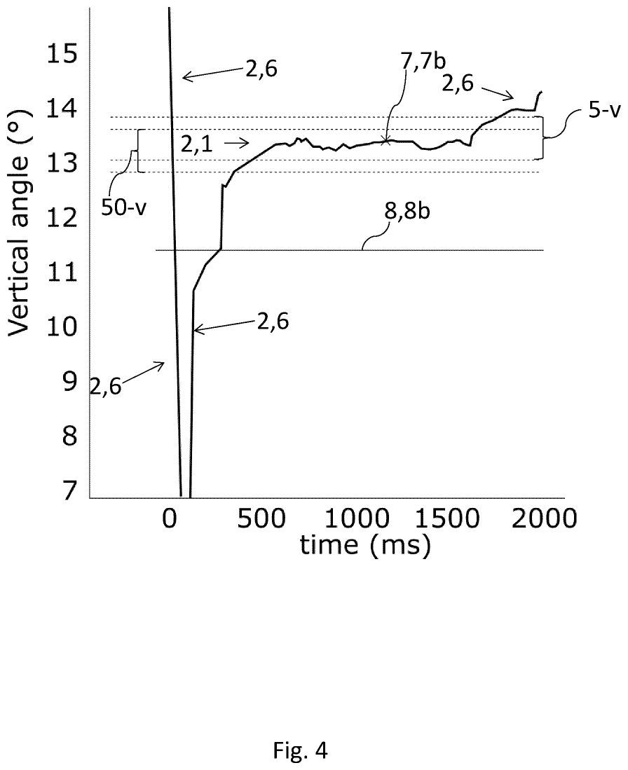

FIG. 4 shows the embodiment of the method according to the invention, wherein an aggregate gaze coordinate is determined from inliers of a second time series of gaze positions of the person in response to an optical stimulus displayed at a second stimulus position shown in FIG. 1;

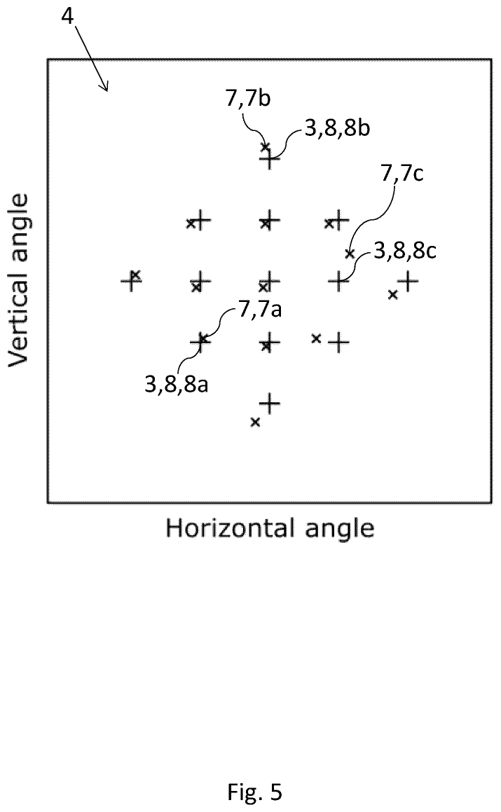

FIG. 5 shows an embodiment of the method according to the invention, wherein determined aggregate gaze coordinates are depicted together with their respective stimulus position in the visual field of the person; and

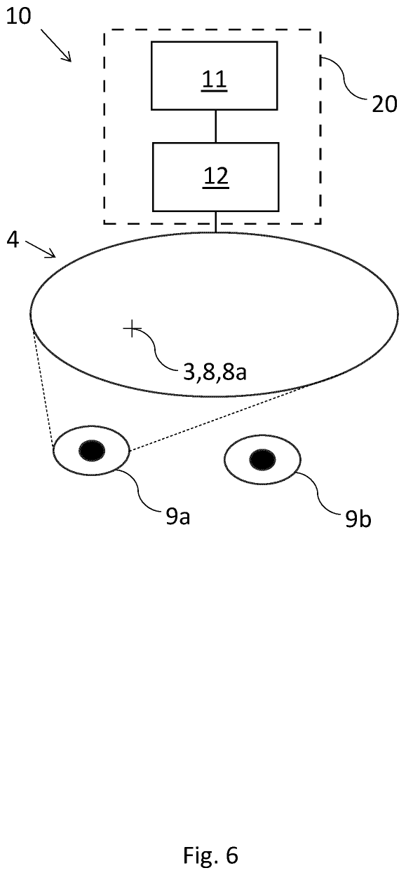

FIG. 6 shows a system for determining inliers of gaze positions of a person, according to an embodiment of the invention.

FIG. 1 shows a plurality of optical stimuli 3 arranged at different stimuli positions 8 within a visual field 4 of a person that can be used to determine inliers 1 of gaze positions 2 of a person from a time series of gaze positions 2 of the person detected in response to the display of individual optical stimuli 3, for example as shown in FIG. 2, FIG. 3 and/or FIG. 4.

To this end, a system 10 according to the invention may be used, which may particularly comprise goggles 10 such as a head-mounted display to be worn by the person, for example as shown in FIG. 6.

In the embodiment of FIG. 1, the optical stimulus 3 is first displayed at a first stimulus position 8,8a for a predetermined time in the visual field 4 of the person, wherein during display of the optical stimulus 3 at the first stimulus position 8,8a, a first time series of gaze positions 2 of the person in the visual field 4 in response to the optical stimulus 3 is detected. Most of the detected gaze positions 2 are located within a first portion 4a of the visual field 4 in proximity to the first stimulus position 8a, cf. FIG. 2. For example, the predetermined time is between 0.1 s and 10 s.

Upon expiry of said predetermined time, the optical stimulus 3 is no longer displayed at the first stimulus position 8,8a but at a different, second stimulus position 8b for said predetermined time in the visual field 4 of the person, wherein during display of the optical stimulus 3 at the second stimulus position 8b, a second time series of gaze positions 2 of the person in the visual field 4 in response to and associated to the optical stimulus 3 displayed at the second stimulus position 8b is detected.

The optical stimulus 3 can subsequently be displayed at a third stimulus position 8c and the remaining stimuli positions 8 of a pattern of stimuli positions shown in FIG. 1, wherein for each stimulus position 8, the associated time series of gaze positions 2 is detected.

FIG. 2 shows a first portion 4a of the visual field 4 in proximity to the first stimulus position 8a. Each dot represents an individual gaze position 2 of the person that is detected upon displaying of the optical stimulus 3 at the first stimulus position 8a. The invention allows to determine inliers 1 of the detected gaze positions 2 that are located within a first range of positions 5, wherein a number of detected gaze positions 2 in the first range 5 is greater than a number of detected gaze positions 2 that are located within any other range of the same given size as the first range 5. In this exemplary case, the first range of positions 5 takes on the shape of an ellipse, which given size may be determined by the orientation and length of its principal axes. However, also other geometrical shapes may be used to define the first range of positions 5, for example a circle, a rectangle and the like. The detected gaze positions 2 comprised by the first range of positions 5 define the inliers 1, wherein the detected gaze positions 2 that are not comprised by the first range of positions 5 define outliers 6. The geometrical shape and the given size of the geometrical shape, i.e. its area in the two-dimensional visual field 4, may be adapted according to a positional spread of the detected gaze positions 2, for example a spatial variance of the detected gaze positions 2. In particular, the given size of the geometrical shape may be larger for a larger positional spread (for example larger variance) of the detected gaze positions 2 compared to smaller positional spread (for example smaller variance) of the detected gaze positions 2. In the angular coordinate system of the visual field 4 shown in FIG. 2, the inliers 1 associated to the first range of positions 5 are spread over a vertical first range of positions 5-v along the vertical axis of the visual field 4 and over a horizontal first range of positions 5-h along the horizontal axis of the visual field 4.

FIG. 2 also shows a second range of positions 50 that has the same given size as the first range of positions 5, i.e. the second range of positions 50 also forms an ellipse in the visual field 4. Additionally, the ellipse associated to the second range of positions 50 comprises the same orientation in the visual field 4 as the ellipse associated to the first range of positions 5. However, the second range of positions 50 is vertically shifted with respect to the first range of positions 5, such that a vertical second range of positions 50-v of the second range of positions 50 does not coincide with the vertical first range of positions 5-v of the first range of positions 5, while a horizontal second range of positions 50-h of the second range of positions 50 does coincide with the horizontal first range of positions 5-h of the first range of positions 5. As a result, and as will be further demonstrated in FIG. 3, the number of detected gaze positions 2 that are located within the second range of positions 50 is lower than the number of detected gaze positions 2 within the first range of positions 5, such that the inliers 1 defined by the second range of positions 5 still represent a good representation of the visual focus of the person. According to the invention, the number of detected gaze positions 2 in the second range of positions 50 amounts to at least 0.5 times the number of gaze positions 2 in the first range of positions 5.

FIG. 3 shows an embodiment of the method according to the invention, wherein a first aggregate gaze coordinate 7a is determined from the inliers 1 of the first time series of gaze positions 2. The first time series of gaze positions 2 is shown in FIG. 2 and corresponds to time-dependent, detected gaze positions 2 of the person in response to the display of the optical stimulus 3 at the first stimulus position 8a of FIG. 1.

In the embodiments of FIG. 1 to FIG. 5, the detected gaze positions 2 are exemplarily represented in a two-dimensional coordinate system with angular coordinates parametrizing the visual field 4, wherein the y-axis in FIG. 3 indicates a vertical angle corresponding to a vertical component of the gaze position 2 of the person in the visual field 4, with respect to a predefined origin of the visual field 4. In the same fashion, a horizontal angle corresponding to a horizontal component of a gaze position of the person in the visual field 4 can be determined (not shown), so as to constitute the full, two-dimensional spatial information of the detected time-dependent gaze positions 2 within the visual field 4, as plotted in FIG. 2. Instead of angles, also other parameters may be used to parametrize the visual field 4, for example by using cartesian coordinate system.

As can be better understood from the temporal representation of the detected gaze positions 2 shown in FIG. 3 than in the spatial plot of the same detected gaze positions 2 of FIG. 2, the first time series of detected gaze positions 2 comprises a positional spread of the detected gaze positions 2 in time, which may be associated to lack of attention of the person or movements between a head mounted display for displaying the optical stimulus 3 and the eye of the person. Nevertheless, the method according to the present embodiment allows to determine inliers 1 of the detected gaze positions 2 that still provide an accurate representation of the visual focus of the person. As a consequence of the large positional spread of the second time series of detected gaze positions 2, some of the inliers 1 are temporally separated by outliers 6. The outliers 6 are located outside the range of positions 5 comprising the inliers 1, cf. also FIG. 2.

FIG. 3 demonstrates the robustness of the method according to the present embodiment for detected gaze positions 2 with relatively large spatial variations of the gaze as a function of time.

Typically, an offset is found between a stimulus position 8 and the detected gaze positions 2, as is also the case between the first stimulus position 8a and the detected gaze positions 2 of the first series of gaze positions 2 shown in FIG. 2 and FIG. 3. This offset is typically different for different persons, and for different stimuli positions 8. The offset depends on the individual physiology of the person, such as shape and functionalities of the eye, as well as the spatial orientation of a display displaying the optical stimulus 3 in the visual field 4 with respect to the eye of the person.

In the projection of the detected gaze positions 2 comprised by the first range of positions 5 onto the vertical axis of the visual field 4 depicted in FIG. 3, the corresponding inliers 1 are arranged within the vertical first range of positions 5-v, cf. FIG. 2. Similarly, a projection of the detected gaze positions 2 onto the horizontal axis of the visual field 4 may be considered, by means of the horizontal first range of positions 5-h, cf. FIG. 2. From the now known inliers 2 associated to the first stimulus position 8,8a and the first range of positions 5, a first aggregate gaze coordinate 7,7a indicated in FIG. 3 associated to the first stimulus position 8,8a can be determined. The first aggregate gaze coordinate 7,7a can for example be calculated as the median or the average position, particularly the median or the average coordinate, of the inliers 1 associated with the first stimulus position 8,8a. The first aggregate gaze coordinate 7,7a according to the present embodiment comprises a vertical component and a horizontal component. In other words, the first aggregate gaze coordinate 7,7a represents a position in the two-dimensional visual field 4.

Similarly, the inliers 1 comprised by the second range of positions 50 (cf. FIG. 2) can be used for the determination of an aggregate gaze coordinate 7. FIG. 3 shows the vertical second range of positions 50-v associated to the second range of positions 50, which is shifted with respect to the vertical first range of positions 50-v associated to the first range of positions 5.

As a consequence of the offset between the inliers 1 and the first stimulus position 8,8a that typically varies for different persons and stimuli positions due to different individual physiologies of the person's eye and/or different spatial orientations of the display with respect to the eye of the person, the first aggregate gaze coordinate 7,7a visible in FIG. 2 is likewise offset from the first stimulus position 8,8a. A transformation model can be used to link each stimulus position 8 with the associated aggregate gaze coordinate 7, so as to provide a calibration between each displayed stimulus position 8 and the associated aggregate gaze coordinate 7, which allows for a spatial correction of virtual contents to be displayed in the visual field 4 of the person using for example a head mounted display, which is particularly relevant for augmented- and/or virtual reality applications. For example, the transformation model can be based on polynomials that establish an analytical connection between each displayed stimulus position 8 and the associated aggregate gaze coordinate 7. The transformation model can also be based on homography or neuronal networks. Since the determination of the aggregate gaze coordinate 7 based on the inliers 1 yields a single aggregate gaze coordinate 7 associated to each stimulus position 8, the transformation model is substantially simplified compared to methods of the prior art that construct the transformation model based on time series of detected gaze positions for each stimulus position 8. For example, if polynomials are used for the transformation model, a second order polynomial is typically sufficient to link each stimulus position 8 to the associated aggregate gaze coordinate 7, which substantially reduces the time needed to generate the transformation model.

FIG. 4 shows an embodiment of the method according to the invention, wherein a second aggregate gaze coordinate 7,7b is determined from inliers 1 of a second time series of gaze positions 2. The second time series of gaze positions 2 corresponds to time-dependent, detected gaze positions 2 of the person in response to the display of the optical stimulus 3 at the second stimulus position 8,8a of FIG. 1.

Upon display of the optical stimulus 3 at the first optical stimulus position 8,8a, the person directs his gaze in a saccadic movement of at least one eye towards the first stimulus position 8,8a, characterized by a relatively large change of the vertical angle of the detected gaze position 2, whereafter the detected gaze position changes on a much smaller scale, once the gaze of the person is directed onto the first optical stimulus position 8,8a.

Compared to the gaze positions 2 of the first time series of gaze positions 2 shown in FIG. 3, the positional spread of the detected gaze positions 2 upon the saccadic movement is smaller, such that the accuracy of a second aggregate gaze coordinate 7b determined from the second time series of gaze positions is larger than for the first time series of detected gaze position 2. The second aggregate position 7b can be determined in the same fashion as explained for FIG. 3.

However, the inliers 1 may also be selected from detected gaze positions 2 that are located within a second range of positions 50, wherein a number of detected gaze positions 2 in the second range of positions 50 amounts to at least 0.5 times the number of gaze positions 2 in the first range of positions 5. An example for such a second range of positions 50 with a slightly lower number of detected gaze coordinates 2 that still exceeds 50% of the number of detected gaze positions 2 located within the first range of positions, particularly the vertical first range of positions 5-v is indicated in FIG. 3. While this approach leads to slightly less robust results compared to selecting the inliers 1 located within the first range of positions 5, the results for the second range of positions 50 may be still good enough for a reliable determination of the aggregate gaze coordinate 7.

FIG. 5 shows an embodiment of the method according to the invention, wherein the optical stimulus positions 8 from FIG. 1 are indicated together with their respective associated aggregate gaze coordinates 7. The aggregate gaze coordinates 7 are each determined from said time series of detected gaze positions 2 of the person in response to the display of the optical stimulus 3 at a specific stimulus position 8 in the visual field 4. For example, the first aggregate gaze coordinate 7a is determined from the first time series of gaze positions 2 detected in response to the display of the optical stimulus 3 at the first stimulus position 8a as shown in FIG. 3 and the second aggregate gaze coordinate 7b is determined from the second time series of gaze positions 2 detected in response to the display of the optical stimulus 3 at the second stimulus position 8b as shown in FIG. 4.

Displacements between the respective stimulus positions 8 and their associated aggregate gaze coordinates 7 can be indicative for a validity of the respective aggregate gaze coordinate 7. For example, if the displacement between a stimulus position 8 and its associated aggregate gaze coordinate 7 exceeds a maximum displacement, this indicates that the determined aggregate gaze coordinate 7 fails to accurately reflect a stabilized gaze direction of the person associated with the stimulus position 8, for example because too strong variations of the detected gaze positions 2 due to distraction of the person or movements of the head mounted display with respect to the eye of the person render the determination of an accurate aggregate gaze coordinate 7 impossible. Aggregate gaze coordinates 7 with displacements that exceed the maximum displacement are preferably discarded and not considered for the transformation model, which improves the accuracy of the transformation model. For example, a third aggregate gaze coordinate 7a associated with the third optical stimulus position 8a as shown in FIG. 5 is discarded, wherein the remaining stimulus positions 8 and their respective associated aggregate gaze coordinate 7 are used for the transformation model. For example, the maximum displacement is determined from an average displacement between stimulus positions 8 and their respective associated aggregate gaze coordinate 7, wherein stimulus positions 8 and their respective associated aggregate gaze coordinate 7 are discarded if their displacement exceeds the average displacement for instance by more than 50%.

Particularly, a succeeding stimulus position 8 can be based on the spatial displacement determined between a preceding stimulus position 8 and the associated aggregate gaze coordinate 7. For example, if for the preceding stimulus position 8 and the associated aggregate gaze coordinate 7, the displacement exceeds the maximum displacement, at least one of the succeeding stimulus positions 8 can be arranged in a vicinity of the preceding stimulus position 8 or again at the preceding stimulus position 8. For example, the vicinity is a circular space in the visual field 4 of the person with a radius of between 20° and 0°, particularly between 5° and 0° around the preceding position. This measure represents a confirmation measurement that can confirm whether the aggregate gaze coordinate 7 associated to the preceding stimulus position 8 was based on an event such as distraction of the user or movements between the head mounted display and the eye of the person, or on systematic problems such as impairments in the visual field 4 of the person or functional issues of the head mounted display. Succeeding stimulus positions can also be based on the spatial displacement determined between a preceding stimulus position 8 and the associated aggregate gaze coordinate 7 in that if a trend between stimulus positions 3 and their associated spatial displacements is found. For example, if for stimulus positions 3 with larger distances to the origin of the visual field 4 the spatial displacement to their respective aggregate gaze coordinates 7 is larger than for stimulus positions 3 with smaller distances to the origin of the visual field 4, at least some of the succeeding stimulus positions can be arranged closer to the origin of the visual field 4 compared to the stimulus positions with larger distances to the origin of the visual field 4. This measure particularly allows to obtain a sufficient number of aggregate gaze coordinate 7 that can be used for the transformation model, even for persons with impaired visual field 4 or problems related to a head mounted display for displaying the optical stimuli 3.

The displacement may be indicated by means of a displacement vector connecting the aggregated gaze coordinates with their respective associated stimulus positions.

FIG. 6 shows a system 10 for determining inliers 1 of gaze positions 2 of a person according to an embodiment of the invention. The system 10 comprises a computer 11 as well as an optical device 12 that are, according to the present embodiment, integrated in or on the goggles 20 to be worn by the person. The goggles can be a head mounted display.

Based on instructions of a computer program executed by the computer 11, the optical device 12 is configured to display the optical stimulus 3 in the visual field 4 of the person, for example as shown in FIG. 1. By means of an example, FIG. 5 depicts the optical stimulus 3 at the first stimulus position 8a in the visual field 1 of the left eye 9a of the person. The right eye 9b of the person may or may not be exposed to the optical stimulus 3. In particular, first one of the eyes 9a, 9b of the person may be exposed to optical stimulus 3, particularly multiple optical stimuli 3 displayed subsequently at different stimulus positions 8, whereafter the other of the two eyes 9b, 9a of the person is exposed to the optical stimulus 3 at multiple stimulus positions 8, so as to determine the inliers 1 for both eyes 9a,9b separately. The optical device 12 is further configured to detect a time series of gaze positions 2 of the person in the visual field 4 in response to the optical stimulus 3.

The gaze positions 2 detected by the optical device 12 are processed by the computer 11 in order to determine the inliers 2 and particularly the aggregate gaze coordinates 7, for example according to the embodiments of FIGS. 1 to 5.

### LIST OF REFERENCE SIGNS

Inliers 1
Gaze positions 2
Optical stimulus 3
Visual field 4
Portion of the visual field 4a
First range of positions 5
Vertical first range of positions 5-v
Horizontal first range of positions 5-h
Outliers 6
Aggregate gaze coordinate 7
First aggregate gaze coordinate 7a
Second aggregate gaze coordinate 7b
Third aggregate gaze coordinate 7c
Stimulus position 8
First stimulus position 8a
Second stimulus position 8b
Third stimulus position 8c
Left eye 9a
Right eye 9b
System 10
Computer 11
Optical device 12
Goggles 20
Second range of positions 50
Horizontal second range of positions 50-h
Vertical second range of positions 50-v

1. A method for determining inliers (1) of gaze positions (2) of a person, wherein the method comprises the steps of:
i) displaying an optical stimulus (3) in a visual field (4) of the person,
ii) detecting a time series of gaze positions (2) of the person in the visual field (4) in response to the optical stimulus (3),
iii) determining inliers (1) of the detected gaze positions (2), wherein the inliers (1) are detected gaze positions (2) that are located within a first range of positions (5), wherein a number of detected gaze positions (2) in the first range (5) is greater than a number of detected gaze positions (2) that are located within any other range of the same given size as the first range (5), or wherein the inliers (1) are detected gaze positions (2) that are located within a second range of positions (50), wherein a number of detected gaze positions (2) in the second range (50) amounts to at least 0.5 times the number of gaze positions (2) in the first range (5), wherein the first and second range of positions (5,50) are found using continuous or discrete optimization techniques or wherein the first and second range of positions (5,50) are found by a sampling technique,
wherein the optical stimulus (3) is subsequently displayed and located for a predetermined time at a plurality of different stimulus positions (8,8a,8b,8c) in the visual field (4) of the person, and wherein for at least some of the stimulus positions (8,8a,8b,8c), respective inliers (1) are determined from respective time series of gaze positions (2).

2. The method according to claim 1, wherein the number of detected gaze positions (2) in the second range (50) amounts to at least 0.7 times the number of gaze positions (2) in the first range (5).

3. The method according to claim 1, wherein detected gaze positions (2) that are not comprised by the first range (5) or the second range of positions define outliers (6) and wherein at least some of the inliers (1) are temporally separated by outliers (6).

4. The method according to claim 1, wherein the given size of the first range of positions (5) is based on a positional spread of the detected gaze positions (2).

5. The method according to claim 4, wherein the given size of the first range of positions (5) is larger for larger positional spread compared to smaller positional spread.

6. The method according to claim 1, wherein during display, the optical stimulus (3) is located for a predetermined time at a predefined stimulus position (8,8a,8b,8c) in the visual field (4) of the person during detection of the time series of gaze positions (2).

7. The method according to claim 1, wherein for at least one stimulus position (8,8a,8b,8c), from the inliers (1) associated to the stimulus position (8,8a,8b,8c), an aggregate gaze coordinate (7,7a,7b,7c) is determined from the inliers (1).

8. The method according to claim 7, wherein for at least one stimulus position (8,8a,8b,8c), a spatial displacement between the stimulus position (8,8a,8b,8c) and the aggregate gaze coordinate (7,7a,7b,7c) associated with the stimulus position (8,8a,8b,8c) is determined.

9. The method according to claim 8, wherein a subsequent stimulus position (8,8a,8b,8c) is based on the spatial displacement between a preceding stimulus position (8,8a,8b,8c) and the aggregate gaze coordinate (7,7a,7b,7c) associated with the preceding stimulus position, and/or wherein the subsequent stimulus position (8,8a,8b,8c) is based on the number and/or the spatial spread of the inliers (1) associated to the preceding stimulus position.

10. The method according to claim 7, wherein a transformation model is generated that links each stimulus position (8,8a,8b,8c) with the associated aggregate gaze coordinate (7,7a,7b,7c).

11. The method according to claim 10, wherein stimulus positions (8,8a,8b,8c) that are associated with a spatial displacement that exceeds a maximum spatial displacement, are not considered for the generation of the transformation model.

12. The method according to claim 1, wherein the optical stimulus (3) is applied to only one eye (9a,9b) of the person and wherein the inliers (1) are determined for the one eye (9a,9b) or for both eyes (9a,9b) separately.

13. The method according to claim 1, wherein the given size is a given input size.

14. The method according to claim 1, wherein the sampling technique comprises sampling the detected gaze positions (2) in a plurality of ranges of positions, particularly in all ranges of positions, to identify the first range of positions (5) or the second range of positions (50).

15. The method according to claim 13, wherein the inliers (1) are determined by sampling the detected gaze positions (2) in the plurality of ranges of positions, particularly in all ranges of positions, wherein each range of positions comprises the same given input size.

16. A computer program for determining inliers (1) of gaze positions (2) of a person, wherein the computer program comprises instructions which, when the program is executed by a computer (11), cause the computer (11) to:
cause an optical device (12) to
display an optical stimulus (3) in a visual field (4) of the person, wherein the optical stimulus (3) is subsequently displayed and located for a predetermined time at a plurality of different stimulus positions (8,8a,8b,8c) in the visual field (4) of the person,
detect a time series of gaze positions (2) of the person in the visual field (4) in response to the optical stimulus (3)
determine inliers (1) of the detected gaze positions (2), wherein the inliers (1) are detected gaze positions (2) that are located within a first range of positions (5), wherein a number of detected gaze positions (2) in the first range (5) is greater than a number of detected gaze positions (2) that are located within any other range of the same given size as the first range (5), or wherein the inliers (1) are detected gaze positions (2) that are located within a second range of positions (50), wherein a number of detected gaze positions (2) in the second range (50) amounts to at least 0.5 times the number of gaze positions (2) in the first range (5), wherein the first and second range of positions (5,50) are found using continuous or discrete optimization techniques or wherein the first and second range of positions (5,50) are found by a sampling technique, wherein for at least some of the stimulus positions (8,8a,8b,8c), respective inliers (1) are determined from respective time series of gaze positions (2).

17. A system (10) for determining inliers (1) of gaze positions (2) of a person, wherein the system (10) comprises:
an optical device (12) configured to:
display an optical stimulus (3) in a visual field (4) of the person,
detect a time series of gaze positions (2) of the person in the visual field (4) in response to the optical stimulus (3), and
the computer (11) according to claim 16.
